(19) United States
(12) Patent Application Publication
(10) Pub. No.: US 2008/0166323 A1
(43) Pub. Date: Jul. 10, 2008

(54) Activators of Hexosamine Biosynthesis as Inhibitors of Injury Induced by Ischemia or Hermorrhagic Shock

(75) Inventors: Richard Marchase, Birmingham, AL (US); Pam Bounelis, Birmingham, AL (US); John Chatham, Birmingham, AL (US); Irshad Chaudry, Birmingham, AL (US); Yi Pang, Chicago, IL (US)

Correspondence Address: NEEDLE & ROSENBERG, P.C., SUITE 1000, 999 PEACHTREE STREET, ATLANTA, GA, 30309-3915, US

(73) Assignee: UAB RESEARCH FOUNDATION, Birmingham, AL (US)

(21) Appl. No.: 10/593,417

(22) PCT Filed: Apr. 14, 2005
(86) PCT No.: PCT/US2005/012547

§ 371 Date: Nov. 30, 2007

Related U.S. Application Data
(60) Provisional application No. 60/562,336, filed on Apr. 14, 2004.

(51) Int. Cl. A61K 35/12; A61K 31/70; A61K 31/7008; A61B 17/00; A01N 1/02; A61K 31/164

(52) U.S. Cl. 424/93.7; 514/25; 514/62; 514/563; 435/1.1; 128/898

(57) ABSTRACT

Disclosed herein are compositions and methods for preserving cell, tissue, or organ transplants and cultures by increasing the concentration of an intracellular metabolite of the hexosamine biosynthetic pathway. Also described herein are methods of reducing pathogenic effects in a subject by increasing the concentration of an intracellular metabolite of the hexosamine biosynthetic pathway.

### CROSS-REFERENCE TO RELATED APPLICATIONS

This application claims benefit of priority to U.S. Provisional Application No. 60/562,336, filed Apr. 14, 2004. U.S. Provisional Application No. 60/562,336 is incorporated by reference herein in its entirety.

### STATEMENT REGARDING FEDERALLY SPONSORED RESEARCH

The research described herein was supported by the National Institutes of Health, grant numbers R01DK55647 and R01HL76175. The U.S. Government may have certain rights in this invention.

### FIELD

The disclosed subject matter relates to methods of preserving cell, tissue, or organ transplants and cultures by increasing the concentration of intracellular metabolites of the hexosamine biosynthetic pathway. The disclosed subject matter also relates to methods of reducing pathogenic effects in a subject by increasing the concentration of intracellular metabolites of the hexosamine biosynthetic pathway.

### BACKGROUND

The Hexosamine Biosynthetic Pathway (HBP) is the process by which glucose is converted into the sugar nucleotide uridine diphosphate-N-acetylglucosamine (UDP-GlcNAc), a substrate in major glycosylation reactions (FIG. 1A). Normally, approximately 2-4% of the glucose (Glc) transported into a cell enters the HBP (Hassell, et al. Ann Rev Biochem (1986) 55:539-567). Once inside the cell, glucose is phosphorylated and converted into fructose-6-phosphate (Fru-6-P). Fructose-6-phosphate can also be generated in the cell by the dephosphorylation of fructose-1,6,-bisphosphate (FBP) with the enzyme fructose-1,6-bisphosphatase. Next, the enzyme glutamine:fructose-6-phosphate amidotransferase (GFAT) catalyzes the conversion of fructose-6-phosphate to glucosamine-6-phosphate (GlcNH₂-6-P) with concomitant conversion of glutamine (Gln) to glutamate. GFAT is the rate-limiting enzyme in the HBP, and flux through the HBP can be inhibited with an amidotransferase inhibitor such as azaserine (Marshall, et al., J Biol Chem (1991) 266(8):4706-4712).

Glucosamine (GlcNH₂), although normally present at low levels in bodily fluids, is also involved in the HBP. Specifically, glucosamine enters cells via glucose transporters (Uldry, et al., FEBS Lett (2002) 524(1-3):199-203) and is phosphorylated to glucosamine-6-phosphate by hexokinase. Glucosamine-6-phosphate, produced either by the GFAT-catalyzed conversion of fructose-6-phosphate or by the hexokinase-catalyzed phosphorylation of glucosamine, rapidly undergoes a series of transformations to arrive at uridine diphosphate-N-acetylglucosamine (UDP-GlcNAc).

UDP-GlcNAc is a key component of the HBP because it is used in the glycosylation of lipids and proteins. For example, a large number of nuclear and cytoplasmic proteins, including transcription factors, cytoskeletal components, signaling components, and enzymes, are modified on a threonine or serine amino acid residue by O-glycosylation with N-acetylglucosamine (GlcNAc). These protein modifications are catalyzed by the enzyme O—N-acetylglucosamine transferase (O-GlcNAc transferase), which uses UDP-GlcNAc as a donor-substrate and releases the byproduct uridine diphosphate (Wells, et al., Science (2001) 291(5512):2376-2378) (FIG. 1B). This modification is distinct from the well-studied glycosylation cascades within the endoplasmic reticulum (ER) and Golgi apparatus, and utilizes completely distinct proteins as acceptors. Like phosphorylation, this modification is reversible, and under at least certain conditions the number of proteins with O-GlcNAc residues within the cell is comparable to the number of phosphorylated proteins (Wells, et al., Science (2001) 291(5512):2376-2378).

As noted, the addition of GlcNAc to proteins with UDP-GlcNAc and O-GlcNAc transferase is reversible. The reverse reaction, the removal of GlcNAc from proteins, is facilitated by the enzyme O—N-acetylglucosaminease (O-GlcNAcase). The dynamic nature of these two competing reactions, addition and removal of GlcNAc, suggests that such protein modifications are an important part of a regulatory mechanism. However, only until recently have researchers begun to understand the HBP and the role of protein glycoconjugates involving GlcNAc (Wells and Hart, FEBS Let, (2002) 546(1):154-158).

Needed in the art are methods and compositions for activating the HBP. Further, methods and compositions of activating the HBP to protect cells, tissues, organs, and patients from damage and to reduce pathogenic effects are also needed. The methods and compositions disclosed herein meet these needs.

### SUMMARY

In accordance with the purposes of the disclosed compositions and methods, as embodied and broadly described herein, in one aspect, the disclosed subject matter relates to methods of preserving cell, tissue, or organ transplants and cultures by increasing the concentration of an intracellular metabolite of the hexosamine biosynthetic pathway. Also described herein are methods of reducing pathogenic effects in a subject by increasing the concentration of an intracellular metabolite of the hexosamine biosynthetic pathway.

Additional advantages will be set forth in part in the description which follows, and in part will be understood from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 is a schematic of the Hexosamine Biosynthetic Pathway (HBP). Panel A shows the pathways involved in the production of uridine diphosphate-N-acetylglucosamine. Panel B shows the pathways involved in the production of protein glycoconjugates from uridine diphosphate-N-acetylglucosamine. In the schemes, Glc is glucose, Glc-6-P is glucose-6-phosphate, FBP is fructose-1,6-bisphosphate, Fru-6-P is fructose-6-phosphate, Gln is glutamine, GlcNH₂ is glucosamine, GlcNH₂-6-P is glucosamine-6-phosphate, GlcNAc is N-acetylglucosamine, GlcNAc-6-P is N-acetylglucosamine-6-phosphate, GlcNAc-1-P is N-acetylglucosamine-1-phosphate, UDP is uridine diphosphate, UDP-GlcNAc is uridine diphosphate-N-acetylglucosamine, GFAT is glutamine:fructose-6-phosphate amidotransferase, GAPDH is glyceraldehyde-3-phosphate dehydrogenase.

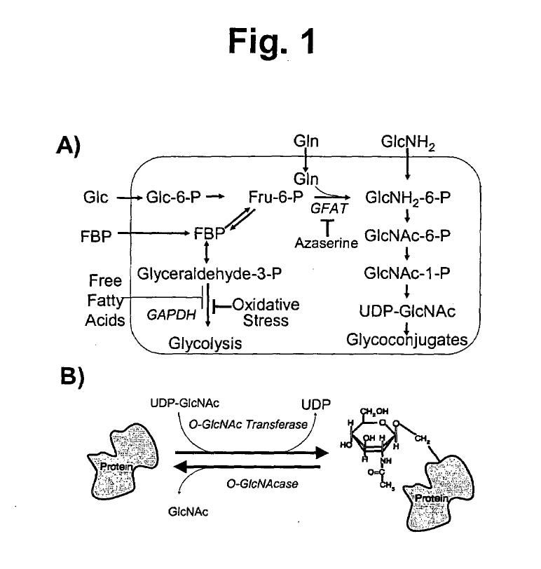

FIG. 2A is a series of graphs of the left ventricular pressure (mm Hg) of hearts during exposure to 10 minutes of Ca²⁺ free perfusate and then 15 minutes of 1.25 mM Ca²⁺ perfusate. “Normal” represents non-hyperglycemic hearts. Hearts made hyperglycemic by treatment with streptozotocin (STZ) are labeled “STZ.” Hearts treated with STZ and azaserine are labeled “STZ+azaserine.” FIG. 2B is a pair of graphs of percent recovery in left ventricular diastolic pressure (LVDP) of normal (labeled “control”) hearts, STZ treated hearts, and STZ plus azaserine treated hearts after readdition of 1.25 mM and 1.8 mM Ca²⁺ perfusate.

FIG. 3A is a graph of percent recovery in LVDP for normal hearts, streptozotocin (STZ) treated hearts, and STZ plus azaserine treated hearts after exposure to 10 minutes of Ca²⁺ free perfusate and then 15 minutes of 1.25 mM Ca²⁺ perfusate. FIG. 3B is a graph of the change in end diastolic pressure (EDP) for normal hearts, STZ treated hearts, and STZ plus azaserine treated hearts after exposure to 10 minutes of Ca²⁺ free perfusate and then 15 minutes of 1.25 mM Ca²⁺ perfusate. FIG. 3C is a graph of protein loss (mg/min/g) from hearts during exposure to 10 minutes of Ca²⁺ free perfusate and then 15 minutes of 1.25 mM Ca²⁺ perfusate. FIG. 3D is a graph of lactate dehydrogenase (LDH) loss (104 B-B Unit/min/g) from hearts during exposure to 10 minutes of Ca²⁺ free perfusate and then 15 minutes of 1.25 mM Ca²⁺ perfusate. For FIGS. 3C and 3D, closed circles represent normal hearts, open circles represent hearts treated with STZ, and open triangles represent hearts treated with STZ plus azaserine.

FIG. 4A is a series of graphs of left ventricular pressure (LVP) (mm Hg) of hearts during exposure to 10 minutes of Ca²⁺ free perfusate and then 15 minutes of 1.25 mM Ca²⁺ perfusate. FIG. 4B is a series of graphs of percent recovery of hearts after exposure to 10 minutes of Ca²⁺ free perfusate and then 15 minutes of 1.25 mM Ca²⁺ perfusate. In FIG. 4B, percent recovery was assessed by rate pressure product (RPP) (RPP=left ventricular pressure (LVP)×heart rate (HR)) (solid circles), +dp/dt (open circles), and LVDP (solid triangles). In the figures, hearts not treated with additional compounds and hearts treated with streptozotocin, glucosamine, the free fatty acid hexanoate, or SKF96365 are respectively labeled “control,” “STZ,” “GlcNH₂,” “FFA,” and “SKF.”

FIG. 5A is a pair of graphs of left ventricular pressure (mm Hg) of hearts during a period of ischemia and reperfusion. The period of ischemia is labeled. The right graph shows data from hearts treated with glucosamine (GlcNH₂) at the time indicated. The left graph shows data from the control. FIG. 5B is a graph of the percent recovery in LVDP before the ischemic period for both control and GlcNH₂ treated hearts. FIG. 5C is a graph of the end diastolic pressure (EDP) (mm Hg) after the ischemic period for both control and GlcNH₂ treated hearts.

FIG. 6 is a pair of graphs of lean and obese Zucker fa/fa rats of 6, 12, and 24 weeks of age. The rats were exposed to a period of 30 minutes of ischemia. The left graph shows left ventricular diastolic pressure (LVDP) as a percentage of pre-ischemic levels. The right graph shows end diastolic pressure (EDP) as a percentage of end ischemic levels.

FIG. 7 is a graph of rate pressure product (RPP) (percent recovery over baseline) of a low-flow isolated rat heart model of ischemia assessed 30 minutes following low-flow perfusion of insulin and 5, 15, and 30 mM of glucose.

FIG. 8 is a photograph of a CTD110 immunoblot performed on extracts from isolated hearts following a 10-minute perfusion with buffer (control) or buffer containing 5 mM glucosamine (GlcNH₂). The differences in the O-GlcNAc-containing protein pattern between the control and the GlcNH₂ treated hearts are indicated by arrows.

### DETAILED DESCRIPTION

The disclosed compositions and methods may be understood more readily by reference to the following detailed description of specific aspects of the materials and methods and the Examples included therein and to the Figures and the previous and following description.

Before the present compositions, methods, articles, and/or devices are disclosed and described, it is to be understood that the aspects described below are not limited to the specifically mentioned components or methods, as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “an intracellular metabolite” includes mixtures of two or more such metabolites; reference to “a stress” includes mixtures of two or more such stresses, reference to “the cell” includes mixtures of two or more cells, and the like.

“Optional” or “optionally” means that the subsequently described event or circumstance may or may not occur, and that the description includes instances where the event or circumstance occurs and instances where it does not.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

As used herein, by a “subject” or “recipient” is meant an individual. Thus, the “subject” or “recipient” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” or “recipient” can also include a mammal, such as a primate or a human.

The terms “higher,” “increases,” “elevates,” or “enhanced” refer to increases above basal levels, e.g., as compared to a control. The terms “lower,” “decreases,” “reduces,” or “reduction” refer to decreases below basal levels, e.g., as compared to a control. By “control” is meant either a subject, organ, tissue, or cell lacking a disease or injury, or a subject, organ, tissue, or cell in the absence of a particular variable such as a therapeutic agent. A subject, organ, tissue, or cell in the absence of a therapeutic agent can be the same subject, organ, tissue, or cell before or after treatment with a therapeutic agent or can be a different subject, organ, tissue, or cell in the absence of the therapeutic agent. Comparison to a control can include a comparison to a known control level or value known in the art. Thus, basal levels are normal in vivo levels prior to, or in the absence of, the addition of an agent (e.g., a therapeutic agent) or another molecule.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

Reference will now be made in detail to specific aspects of the disclosed materials, compounds, compositions, components, and methods, examples of which are illustrated in the accompanying drawing.

Disclosed herein are methods and compositions related to the surprising discovery that intracellular metabolites of the HBP, such as, e.g., glucosamine, glutamine, and fructose-1,6-bisphospate, etc., which amplify or accelerate the increases in GlcNAc addition to proteins, greatly decrease cellular and tissue damage. Further, the beneficial effect of this treatment occurs over a period of minutes to hours, rather than days to weeks and is observed in isolated tissues as well as in vivo, illustrating that the effect is not mediated by inhibition of the immune system.

### Methods of Preserving Cell, Tissue, or Organ Transplants

Disclosed herein are methods of preserving a cell, tissue, or organ transplant in a transplant recipient by contacting the cell, tissue, or organ transplant with a composition that increases a concentration of an intracellular metabolite of a hexosamine biosynthetic pathway, as compared to the concentration of the intracellular metabolite in the absence of the composition; and transplanting the cell, tissue, or organ into the recipient, the increase in the concentration of the intracellular metabolite of the hexosamine biosynthetic pathway preserving the cell, tissue, or organ transplant in the transplant recipient.

The Hexosamine Biosynthetic Pathway (HBP) refers singularly and collectively to the various, specific cellular processes by which UDP-GlcNAc is generated. For example, the HBP includes processes whereby glucose is converted to UDP-GlcNAc, whereby fructose-1,6-bisphosphate is converted to UDP-GlcNAc, and whereby GlcNH₂ is converted to UDP-GlcNAc. The HBP also includes the O-glycosylation of proteins and lipids with GlcNAc, as well as the removal of GlcNAc from proteins and lipids.

Intracellular metabolites of the HBP include, but are not limited to, glucose, glucose-6-phosphate, fructose-6-phosphate, fructose-1,6,-bisphosphate, glutamate, glutamine, glutamine-6-phosphate, N-acetylglucosamine-6-phosphate, N-acetylglucosamine-1-phosphate, and uridine diphosphate N-acetylglucosamine. Similarly, intracellular metabolites of the HBP include, but are not limited to, proteins and lipids that have been modified by O-glycosylation with GlcNAc; these include, but are not limited to, proteins such as heat shock proteins, crystallins, cytoskeleton proteins, transcription factors, and glycolipids such as gangliosides. The concentrations of such intracellular metabolites of the HBP can be measured by methods known to those of skill in the art, such as, but not limited to, high performance liquid chromatography (HPLC), gas chromatography (GC), gas chromatography mass spectrometry (GCMS), nuclear magnetic resonance (NMR), electrophoresis, and the like. (See Methods in Enzymology, volume 230, Guide to Techniques in Glycobiology, edited by W. J. Lennarz and Gerald W. Hart, Academic Press, Inc. 1994, which is incorporated by reference herein for its teachings of methods for analyzing intracellular metabolies.)

Contacting a cell, tissue, or organ transplant with a composition that increases intracellular metabolites of the hexosamine biosynthetic pathway can be performed at any time. For example, the contacting step can be performed prior to the transplantation step. In another example, the contacting step can be performed during the transplantation step. In yet another example, the contacting step can be performed after the transplantation step.

The cell, tissue, or organ transplant can be contacted with a composition that increases a concentration of an intracellular metabolite of the HBP in a variety of ways. For example, the cell, tissue, or organ transplant can be submerged or immersed in the composition. In another example, the cell, tissue, or organ transplant can be coated or sprayed with the composition. In still another example, the cell, tissue, or organ transplant can be contacted with a medium, such as a growth medium, that contains the composition. In a further example, the cell, tissue, or organ transplant can be infused with the composition. The various methods of contacting the cell, tissue, or organ transplant with the compositions disclosed herein will be readily apparent to one of ordinary skill in the art, depending on such factors as the type of cell, tissue, or organ transplant, the particular composition to be used, the condition of the transplant recipient, convenience, and the like.

Any cell, tissue, or organ transplant that produces GlcNAc glycoconjugates via the HBP can be contacted with a composition that increases a concentration of an intracellular metabolite of the HBP. In one example, the cell, tissue, or organ transplant is not hyperglycemic or in a hyperglycemic environment.

A suitable cell for use in the methods described herein can be of any cell type, from any tissue, and from any organism, as long as the cell, tissue, or organ utilizes the HBP. For example, a suitable cell can be derived from any eukaryotic species and can be differentiated, undifferentiated, de-differentiated, or immortalized. The cell can be freshly derived from the eukaryotic species or can be from a primary culture or cultured cell line. In addition, the cell can be genetically modified, e.g., to decrease or eliminate expression of one or more undesirable proteins (e.g., a cell-surface immunogen that would induce an undesirable immune response) or to induce or increase expression of one or more desirable endogenous or foreign proteins (e.g., a therapeutic protein or a marker that can be used to select and/or identify the cell). The cell can be derived from any vertebrate species, including, but not limited to, mammalian cells (such as rat, mouse, bovine, porcine, sheep, goat, and human), avian cells, fish cells, amphibian cells, reptilian cells, and the like. It is also contemplated that a population of cells, containing the same type of cells or a mixture of different types of cells can be used in the methods herein. In this sense, a tissue or organ transplant will likely, but need not, contain a mixture of different cell types.

Some specific examples of the various cell types that can be preserved by the present methods include, but are not limited to, neurons, muscle cells, pancreatic islet/beta cells, cardiocytes, cardiomyocytes, hepatocytes, glomerulocytes, epithelial cells, immune cells (including lymphatic cells, T cells, and B cells), macrophages, eosinophils, neutrophils, stem cells, germ cells (i.e., spermatocytes/spermatozoa and oocytes), fibroblasts, follicular cells, zygotes, embryonic cells, hematopoietic cells, and the like. Such cells can be taken from organisms under normal basal conditions, under naturally occurring or induced disease states or following some sort of activation, stimulation or other perturbation of the organism, including, for example, genetic, pharmacologic, surgical, pathogenic, or therapeutic manipulations.

Examples of tissue that can be contacted with a composition that increases a concentration of an intracellular metabolite of the HBP can be, for example, skin, muscle, bone, vascular, or connective tissues. Examples of organs that can be contacted with a composition that increases a concentration of an intracellular metabolite of the HBP can include, but are not limited to, liver, kidney, spleen, bone marrow, thymus, heart, muscle, lung, testes, ovary, intestine, skin, bone, stomach, pancreas, gall bladder, prostate, and bladder. For example, the organ transplant can be a heart.

The choice of the cell, tissue, or organ transplant can be made by one of ordinary skill in the art. The choice will depend on the particular desires and aims of the researcher or clinician and also the needs of the recipient.

According to a method disclosed herein, a cell, tissue, or organ transplant is contacted with a composition that increases a concentration of an intracellular metabolite of the HBP. Suitable compositions are those that increase the concentration of an intracellular metabolite of HBP. In one aspect, the composition can increase the concentration of one or more intracellular metabolites of HBP. For example, a suitable composition can include any combination of glucosamine, N-acetylglucosamine, glutamine, or fructose-1,6-bisphosphate, or a pharmaceutically acceptable salt or a polymer thereof.

The compositions used herein are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). These references are incorporated by reference herein for their teachings of synthetic methods and procedures.

In one aspect, a composition that can be contacted with a cell, tissue, or organ transplant to increase a concentration of an intermediate of the HBP includes, but is not limited to, glucosamine, glucosamine polymer, N-acetylglucosamine polymer, or a pharmaceutically acceptable salt thereof. In another example, the composition includes N-acetylglucosamine, N-acetylglucosamine polymer, or a pharmaceutically acceptable salt thereof.

Examples of glucosamine and N-acetylglucosamine polymers include, but are not limited to, chitosan and chitin. Chitosan is a naturally occurring polymer found in many fungi. However, as a matter of convenience, chitosan is obtained from chitin, which (after cellulose) is the second most abundant natural polymer. Chitin is readily isolated from shellfish or insect exoskeletons, and is also found in mollusks and fungi. Chitin is a water-insoluble copolymer of N-acetyl-D-glucosamine and D-glucosamine, but the great preponderance of monomer units are N-acetyl-D-glucosamine residues. Chitosan is a copolymer of the same two monomer units, but the preponderance of monomer units are D-glucosamine residues. Since the D-glucosamine residues bear a basic amino function, they readily form salts with acids. Many of these salts are water soluble. Treatment of chitin with concentrated caustic at elevated temperature converts N-acetyl-D-glucosamine residues into D-glucosamine residues and thereby converts chitin into chitosan. There is a continuum of compositions possible between pure poly-N-acetyl-D-glucosamine and pure poly-D-glucosamine. These compositions are all within the skill of the art to prepare and are all suitable for the uses described herein.

Suitable acids for making the chitosan salts for use in the methods described herein are those acids that form water-soluble salts with chitosan. It is not necessary that the acid itself be water-soluble; however, such water-soluble acids can ease handling. Inorganic acids, which form water-soluble chitosan salts, include the halogen acids and nitric acid, but exclude sulfuric and phosphoric acids because they do not form water-soluble salts with chitosan. Organic acids are particularly suitable and include, but are not limited to, lactic acid, glycolic acid, glutamic acid, formic acid, acetic acid, and a mixture thereof. Either mono- or poly-functional carboxylic acids can also be used. They can be aliphatic or aromatic, so long as they form water-soluble salts with chitosan.

In yet another aspect, a composition that can be contacted with a cell, tissue, or organ transplant to increase a concentration of an intermediate of the HBP includes, but is not limited to, glutamine or a pharmaceutically acceptable salt thereof. Glutamine is a necessary substrate for GFAT and, as noted above, is required for glucose flux through the HBP. Increasing glutamine concentrations increases UDP-GlcNAc, especially if glucose levels are also elevated (Wu, et al., Biochem J (2001) 353(Pt 2):245-252). Elevating glutamine above the normal plasma level of about 0.4 mM has been shown to enhance recovery from a variety of experimental and clinical challenges including burns, sepsis, and trauma (reviewed in Wischmeyer, et al., JPEN J Parenter Enteral Nutr (2003) 27(2):116-122). With respect to cardiac ischemia, glutamine provides remarkable protection either when administered prior to ischemia or when administered just prior to reperfusion in an isolated rat heart (Khogali, et al., J Mol Cell Cardiol (1998) 30(4):819-827; Khogali, et al., Nutrition (2002) 18(2):123-126). Glutamine was also protective in an isolated cardiomyocyte model (Wischmeyer, et al., JPEN J Parenter Enteral Nutr (2003) 27(2): 116-122). To date, however, the benefit of enhancing flux through the HBP were not appreciated.

In still another aspect, a composition that can be contacted with a cell, tissue, or organ transplant to increase a concentration of an intermediate of the HBP includes, but is not limited to, fructose-1,6-bisphosphate (FBP) or a pharmaceutically acceptable salt thereof. There is extensive literature documenting that FBP is protective in animal models of ischemia/reperfusion (Woo, et al., Heart Surg Forum (2003) 6 Supp 1:S36; Lazzarino, et al., Free Radic Res Commun (1992) 16(5):325-339) and hemorrhagic shock (Granot and Snyder, Proc Natl Acad Sci USA (1991) 88:5724-5728). FBP also leads to improved outcomes in human studies (Markov, et al., Am Heart J (1997) 133(5):541-549). The postulated mechanism of action is that provision of FBP leads to an increase in glycolytic ATP production (Hardin, et al., Am J Physiol Heart Circ Physiol (2001) 281(6):H2654-H2660). It is initially surprising that this doubly phosphorylated sugar should permeate the plasma membrane, but recent data are consistent with its transport via a dicarboxylate transport system (Hardin, et al., Am J Physiol Heart Circ Physiol (2001) 281(6):H2654-H2660). However, in addition to being a potential fuel for glycolysis, FBP is an important intermediate in the HBP pathway, where it is converted to fructose-6-phosphate by fructose-1,6-bisphosphatase. As illustrated by GAPDH inhibition, elevated FBP, such as that resulting from GAPDH inhibition, will enhance flux through the HBP (Du, et al., Proc Natl Acad Sci USA (2000) 97(22):12222-12226) and lead to an increase in UDP-GlcNAc and protein-associated O-GlcNAc.

The compositions detailed herein, which can be contacted with a cell, tissue, or organ transplant to increase a concentration of an intermediate of the HBP, can be prepared as pharmaceutically acceptable salts, which are also suitable compositions for the disclosed methods. For example, pharmaceutically acceptable salts of glucosamine, N-acetylglucosamine, polymers of glucosamine and/or N-acetylglucosamine, glutamine, and fructose-1,6-bisphosphate can be used. Pharmaceutically acceptable salts can be prepared by treating the compound with an appropriate amount of a pharmaceutically acceptable acid or base. Representative pharmaceutically acceptable acids include, but are not limited to, hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid. Representative pharmaceutically acceptable bases include, but are not limited to, ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C., such as at room temperature. The molar ratio of the compounds to base used is chosen to provide the ratio desired for any particular salts. For preparing, for example, the sodium salts of a composition, the composition can be treated with approximately one equivalent of pharmaceutically acceptable base, e.g., NaOH, to yield a neutral salt.

While not wishing to be bound by theory, the protection seen with the compositions disclosed herein is an increase in protein-associated O-GlcNAc. An alternative strategy to increase protein-associated O-GlcNAc is to inhibit the enzyme responsible for the removal of O-GlcNAc (i.e., O—N-acetyl glucosaminease (O-GlcNAcase)). Therefore, according to the methods disclosed herein, an inhibitor of O-GlcNAcase is a composition that can be used to increase the concentration of an intracellular metabolite of the HBP. An inhibitor of O-GlcNAcase can be used alone or with one or more of the above described compositions. An example of a inhibitor that can be used in the methods disclosed herein is commonly known as PUGNAc, which is O-(2-acetamido-2-deoxy-D-glucopyranosylidene)amino-N-phenylcarbamate. (PUGNAc is commercially available from Carbogen; Aarau, Switzerland; see Arias, et al., Diabetes (2004) 53(4):921-930, which is incorporated herein for its teachings and methods of using PUGNAc.)

The amount of a composition that can be contacted to a cell, tissue, or organ transplant can be any amount that will increase a concentration of an intracellular metabolite of the HBP. For example, the concentration of the composition can be from about 0.1 mM to about 1 M. In another example, the concentration of the composition can be from about 0.1 mM to about 10 mM, from about 1 mM to about 100 mM, or from 10 mM to 1000 mM (1M). In yet another example, the concentration of the composition can be about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, or 100 mM, where any of the stated values can form an upper or lower end point as appropriate.

In a still further example, the concentration of the composition can be about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 (1M), where any of the stated values can form an upper or lower endpoint as appropriate.

The composition can comprise, e.g., glucosamine, glucosamine polymer, or pharmaceutically acceptable salts thereof in a concentration of from about 0.1 mM to about 1 M. Also, the composition can comprise an inhibitor of O-GlcNAcase, such as PUGNAc, in an amount of from about 1 mM to about 1 M, from about 50 mM to about 500 mM, or about 100 mM.

One or more intracellular metabolites of the HBP can have their concentrations increased by contacting a cell, tissue, or organ transplant with the compositions disclosed herein. The particular intracellular metabolites are discussed above, as are methods for measuring the concentration of an intracellular metabolite.

An increase in the concentration of an intracellular metabolite of the HBP can be determined by comparing the concentration of an intracellular metabolite from a cell, tissue, or organ transplant that has been contacted with a composition disclosed herein with the concentration of the same intracellular metabolite from a cell, tissue, or organ transplant that has not been contacted with the composition. The determination of an increase in a concentration of an intracellular metabolite of the HBP can also be made by comparing the concentration of an intracellular metabolite from a cell, tissue, or organ transplant after contact with a composition disclosed herein with the concentration of the same intracellular metabolite from the same cell, tissue, or organ prior to contact with the composition.

The compositions for use in the present methods can increase an intracellular metabolite of the HBP in a cell, tissue, or organ transplant by about 10%, about 25%, about 50%, about 75%, or about 100%, as compared to the intracellular metabolite in the absence of the composition. More specifically, the intracellular metabolite can be increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, where any of the stated values can form an upper or lower endpoint as appropriate.

The increase in concentration of an intracellular metabolite of the HBP can be measured at any time. For example, the increase in the concentration of an intracellular metabolite can be measured at from about 1 minute to about 1 hour after contact with a composition disclosed herein. For example, the increase in the concentration of an intracellular metabolite can be measured from about 5 minutes to about 45 minutes, from about 15 minutes to about 30 minutes, or at about 20 minutes after contact with a composition disclosed herein. In one aspect, the increase in the concentration of an intracellular metabolite of the HBP can be measured at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes after contact with a composition disclosed herein, where any of the stated values can form an upper or lower endpoint as appropriate.

It is contemplated that any of the values stated above for the increase in concentration of an intracellular metabolite can be measured at any of the times stated above after contact with a composition disclosed herein. For example, the concentration of an intracellular metabolite of the HBP can be from about 1 to about 100%, measured from about 1 to about 60 minutes after contact with the composition.

As an example, the concentration of the intracellular metabolite UDP-GlcNAc can be increased by about 75%, 30 minutes after contact with a composition disclosed herein. In another aspect, UDP-GlcNAc can be increased by about 50%, 10 minutes after contact with a composition disclosed herein. In yet another aspect, UDP-GlcNAc can be increased by about 20%, 10 minutes after contact with the composition. As another example, the concentration of the intracellular metabolite glucose-6-phosphate can be increased by about 75%, 30 minutes after contact with a composition disclosed herein. In another aspect, glucose-6-phosphate can be increased by about 50%, 10 minutes after contact with a composition disclosed herein. In yet another aspect, glucose-6-phosphate can be increased by about 20%, 10 minutes after contact with the composition.

The cells, tissues, or organs that have been contacted with the compositions disclosed herein can then be transplanted into a transplant recipient. Methods of transplanting cell, tissues, and organs are known to those skilled in the art.

The increase in concentration of an intracellular metabolite of the HBP preserves the cell, tissue, or organ transplant. “Preservation” or “protection” can be attained by any physiological or therapeutic mechanism that contributes to the conservation of a cell, tissue, organ, or recipient by attenuating damage to the cell, tissue, organ, or recipient. Preservation or protection can be determined by methods known in the art, such as by assaying viability, activity, function, resistance to damage or injury, resilience from damage or injury, and the like. In general, protection or preservation occurs when a cell, tissue, or organ has a higher viability, is more resistant to injury, is more resilient from damage or injury, has increased activity, or has enhanced function as compared to a control cell, tissue, or organ.

### Methods of Preserving Cell, Tissue, or Organ Cultures

Also disclosed herein are methods of preserving a cell, tissue, or organ culture, comprising contacting the cell, tissue, or organ culture with a composition that increases a concentration of an intracellular metabolite of a hexosamine biosynthetic pathway, as compared to the concentration of the intracellular metabolite in the absence of the composition, the increase in the concentration of the intracellular metabolite of the hexosamine biosynthetic pathway preserving the cell, tissue, or organ culture.

Contacting a cell, tissue, or organ culture with a composition disclosed herein can be performed by any of the methods disclosed above for cell, tissue, and organ transplants. Cell, tissue, or organ cultures treated by the methods herein can contain any of the cells, tissues, or organs described herein with regard to cell, tissue, or organ transplants, and/or known in the art.

Further, the compositions that can be used to increase a concentration of an intracellular metabolite of the HBP in a cell, tissue, or organ culture can be any of those described herein for use with cell, tissue, or organ transplants. For example, the composition can comprise any combination of glucosamine, N-acetylglucosamine, glutamine, or fructose-1,6-bisphosphate, or a pharmaceutically acceptable salt or polymer thereof. In another example, the composition can comprise glucosamine, glucosamine polymer, or a pharmaceutically acceptable salt thereof.

Also, the composition can be used in any amount as disclosed above for use with cell, tissue, or organ transplants. For example, the composition can comprise glucosamine, glucosamine polymer, or a pharmaceutically acceptable salt thereof in a concentration of from about 0.1 mM to about 1 M.

The intracellular metabolites of the HBP for cell, tissue, and organ cultures are the same as those disclosed above. Similarly, the increase in concentration of an intracellular metabolite can be increased as disclosed above, and the increase can be measured as disclosed above. For example, the intracellular metabolite UDP-GlcNAc can be increased by about 75%, 30 minutes after contact with a composition disclosed herein. As another example, UDP-GlcNAc can be increased by about 50%, 10 minutes after contact with a composition disclosed herein. In yet another aspect, UDP-GlcNAc can be increased by about 20%, 10 minutes after contact with a composition disclosed herein.

Further, as discussed above for transplants, by contacting the cell, tissue, or organ culture with the compositions disclosed herein, the concentration of an intracellular metabolite of the HBP increases, resulting in the preservation of the cell, tissue, or organ culture.

### Methods of Reducing Pathogenic Effects

Also disclosed herein are methods of reducing pathogenic effects caused by stress in a subject, comprising administering to the subject a composition that increases a concentration of an intracellular metabolite of a hexosamine biosynthetic pathway, as compared to the concentration of the intracellular metabolite in the absence of the composition, the increase in the concentration of the intracellular metabolite of the hexosamine biosynthetic pathway reducing the pathogenic effects of the stress. The subject can be any subject as defined above. For example, the subject can be a mammal, such as a human. In a further example, the subject is not hyperglycemic.

Pathogenic effect means an impairment of the normal state of the living cell, tissue, organ, recipient, or subject, or one of its parts, that interrupts or modifies the performance of one or more vital functions. Pathogenic effects can be caused by environmental factors (such as malnutrition, industrial hazards, or climate), by specific infective agents (such as worms, fungi, bacteria, or viruses), by inherent defects (such as genetic anomalies), by a physical stress, or by combinations of these factors.

Examples of stresses that can cause a pathogenic effect include, but are not limited to, ischemia, hemorrhage, hypovolemic shock, myocardial infarction, stroke, and medical procedures, such as an interventional cardiology procedure, cardiac bypass surgery, fibrinolytic therapy, angioplasty, or stent placement. In another example, the stress is not associated with a hyperactivated inflammatory response.

The compositions that can be used to reduce pathogenic effects caused such stresses in a subject can be any of the compositions disclosed above for preserving cell, tissue, and organ transplants and cultures, i.e., the compositions disclosed above that increase a concentration of intracellular metabolites of the HBP. For example, the composition can comprise glucosamine, N-acetylglucosamine, or a pharmaceutically acceptable salt or a polymer thereof. In another example, the composition can comprise glutamine or a pharmaceutically acceptable salt thereof. In yet another example, the composition can comprise fructose-1,6-bisphosphate or a pharmaceutically acceptable salt thereof. In still another example, the composition can comprises any combination of glucosamine, N-acetylglucosamine, glutamine, fructose-1,6-bisphosphate, or a pharmaceutically acceptable salt or a polymer thereof. In a still further example, the composition can comprise an inhibitor of O-GlcNAcase.

Administration of a composition to a subject in accordance with the methods herein can be accomplished prior to, during, or after a stress. The dosage or amount of the composition should be large enough to produce the desired effect in the method by which delivery occurs; however, the dosage should not be so large as to cause adverse side effects, such as unwanted cross-reactions, anaphylactic reactions, and the like. In this sense, the dosage should be large enough to increase a concentration of an intracellular metabolite of the HBP without adversely affecting the subject. Generally, the dosage will vary with subject to subject, depending on the species, age, weight, sex, general condition, the particular composition being administered, and extent of the disease or stress in the subject, and can be determined by one of skill in the art. The dosage can be adjusted by the individual physician based on the clinical condition of the subject involved. The dose, schedule of doses, and route of administration can also be varied.

The increase in a concentration of an intracellular metabolite of the HBP can reduce the pathogenic effects of a stress. A reduction in pathogenic effects indicates that administration of a given composition has been effective (e.g., in a particular dosage and/or dosage regimen). Such efficacy can be determined by evaluating the particular aspects of the medical history, signs, symptoms, and objective laboratory tests that are known to be useful in evaluating the status of a subject in need of such reduction of pathogenic effects (caused by, for example, stress), or for treatment of other diseases and conditions that can be treated using the methods herein. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group or knowledge of the normal progression of the disease in the general population or the particular individual: 1) a subject's physical condition is shown to be improved (e.g., cardiac outcomes are improved), 2) the progression of the disease or condition is shown to be stabilized, or slowed, or reversed, or 3) the need for other medications for treating the disease or condition is lessened or obviated, then a particular treatment regimen will be considered efficacious.

Any of the compositions disclosed herein can be used therapeutically in combination with a pharmaceutically acceptable carrier. In another aspect, any of the compositions disclosed herein can be used prophylactically, i.e., as a preventative agent, with a pharmaceutically acceptable carrier. The compositions disclosed herein can be conveniently formulated into pharmaceutical compositions composed of one or more of the compositions disclosed herein in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compositions disclosed herein and which is incorporated by reference herein. Such pharmaceutical carriers, most typically, would be standard carriers for administration of compositions to humans and non-humans, including solutions such as sterile water, saline, and buffered solutions at physiological pH. Other compounds will be administered according to standard procedures used by those skilled in the art.

Depending on the intended mode of administration, the pharmaceutical compositions can be in the form of, for example, solids, semi-solids, liquids, solutions, suspensions (e.g., incorporated into microparticles, liposomes, etc.), emulsions, gels, or the like, preferably in unit dosage form suitable for single administration of a precise dosage. The pharmaceutical compositions can include, as noted above, an effective amount of the conjugate in combination with a pharmaceutically acceptable carrier and, in addition, can include other carriers, adjuvants, diluents, thickeners, buffers, preservatives, surfactants, etc. Pharmaceutical compositions can also include one or more active ingredients such as other medicinal agents, pharmaceutical agents, antimicrobial agents, anti-inflammatory agents, anesthetics, and the like.

Liquid pharmaceutically administrable compositions can, for example, be prepared by dissolving, dispersing, etc., a composition as described herein and optional pharmaceutical adjuvants in an excipient, such as, for example, water, saline aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example see Remington's Pharmaceutical Sciences, referenced above.

The compounds and pharmaceutical compositions described herein can be administered to the subject in a number of ways depending on whether local or systemic treatment is desired, and on the area to be treated. Thus, for example, a compound or pharmaceutical composition described herein can be administered as perfusion buffer. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein for its teaching of sustained release systems.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives, such as antimicrobials, anti-oxidants, chelating agents, and inert gases and the like, can also be present.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

In one specific aspect, a composition that can increase the concentration of an intracellular metabolite of the HBP is in the form of a solution in Ringer's lactate. For example, the composition can comprise a solution of from about 0.1 mM to about 1 M glucosamine in from about 100% to about 50% Ringer's lactate. In one aspect, the composition can comprise a from about 0.1 mM to about 10 mM, from about 1 mM to about 100 mM, or from 10 mM to 1000 mM (1M) solution in Ringer's lactate. In another aspect, the composition can be in from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, or 50% Ringer's lactate.

The compositions disclosed herein can be administered to a subject continuously over a period of time, in distinct doses over a period of time, or in one dose. The administration regimen can be chosen by one of skill in the art depending on such factors as depending on the species, age, weight, sex, general condition, the particular composition being administered, and extent of the disease or stress in the subject.

In one aspect, the compositions disclosed herein can be administered to a subject in one dose. In another aspect, the compositions disclosed herein can be administered at from about 5 minutes to about 1 hour, from about 10 minutes to about 50 minutes, or from about 20 minutes to about 40 minutes. In yet another aspect, the compositions disclosed herein can be administered for not more than about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 minute, where any of the stated values can form an upper or lower endpoint as appropriate.

As disclosed above, the administration of the compositions disclosed herein can increase a concentration of an intracellular metabolite of the HBP, such as UDP-GlcNAc and glucosamine-6-phosphate. This increase in concentration of an intracellular metabolite can result in the reduction of pathogenic effects caused by stress. Also, the increase in concentration of an intracellular metabolite can inhibits cellular calcium overload.

The disclosed cell, tissue, and organ protection therapies that decrease damage during and following stresses such as ischemia can have profound implications in at least four clinical settings: (1) injuries resulting in hemorrhage and hypovolemic shock; (2) recovery from myocardial infarction or stroke; (3) interventional cardiology procedures such as cardiac bypass, fibrinolytic therapy, and angioplasty/stent placement; and (4) preservation of organs prior to and following transplant.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, desired solvents, solvent mixtures, temperatures, pressures and other reaction ranges and conditions that can be used to optimize the methods described herein. Only reasonable and routine experimentation will be required to optimize such process conditions.

### Example 1

The following experiments were conducted using a model of soft tissue trauma-hemorrhage (T-H) as described in Ba, et al., Crit. Care Med (2000) 28(8):2837-2842; Chaudry, et al., Can J Physiol Pharmacol (1975) 53(5):859-865; Ma, et al., Am J Physiol Gastrointest Liver Physiol (2003) 284(1):G107-G115; and Harkema and Chaudry, Crit. Care Med (1992) 20(2):263-275. Male rats were fasted for 16 h and divided into three groups: (1) sham (trauma only); (2) T-H (trauma with subsequent hemorrhage and resuscitation); and (3) G-T-H (trauma with subsequent hemorrhage and resuscitation in the presence of glucosamine). All groups underwent a 5 cm ventral midline laparotomy to induce soft-tissue trauma and were cannulated in both femoral arteries and the right femoral vein. The T-H and G-T-H groups received an infusion of 3 mls over 30 minutes of Ringer's lactate (RL) containing, in the G-T-H group only, 10 mM glucosamine. These two groups were then bled to a mean arterial pressure of 40 mm Hg within 10 minutes and maintained at that pressure for 80 minutes. The T-H group was then infused with 1 ml RL over 10 minutes, while in the G-T-H group the infusate contained an iso-osmotic solution of 75 mM glucosamine (pH 7.4) in 50% RL. The two groups were then resuscitated with four times the maximum hemorrhage volume over a period of 60 minutes with RL (T-H) or RL containing 10 mM glucosamine (G-T-H). Sham-operated animals underwent the same surgical procedure but were neither bled nor resuscitated. Two hours post-resuscitation, a catheter was inserted into the left ventricle via the right carotid artery. Organ blood flows were determined using radioactive microspheres. Cardiac function and blood parameters were also assessed. The data for these experiments are shown in Tables 1 and 2 (values are mean ±standard error of the mean).

TABLE 1
Alterations in Systemic Hemodynamic Parameters 2 h after T-H

| Assessment Parameter | SHAM (n = 2) | T-H (n = 3) | G-T-H (n = 3) |
|---|---|---|---|
| Cardiac Output | 27.7 ± 1.0 | 16.2 ± 4.7 | 40.5 ± 0.4 |
| (ml/min 100 g | | | |
| body wt) | | | |
| Oxygen Delivery | 5.03 ± 0.3 | 1.35 ± 0.3 | 3.3 ± 0.1 |
| (ml/min 100 g | | | |
| body wt) | | | |
| Oxygen Consumption | 1.24 ± 0.20 | 0.61 ± 0.09 | 1.32 ± 0.15 |
| (ml/min 100 g | | | |
| body wt) | | | |
| Oxygen Extraction | 24.5 ± 2.7 | 50.2 ± 14.1 | 42.0 ± 5.4 |
| (%) | | | |
| Heart Rate | 358 ± 36 | 344 ± 40 | 371 ± 15 |
| (beat/minute) | | | |
| Mean Arterial Pressure | 117 ± 7 | 91 ± 12 | 104 ± 3 |
| (mm Hg) | | | |
| +dp/dt | 12,100 ± 890 | 8,570 ± 800 | 14,400 ± 900 |
| (mm Hg/sec) | | | |
| −dp/dt | 9,600 ± 1,500 | 5,200 ± 1,000 | 8,700 ± 600 |
| (mm Hg/sec) | | | |
| Stroke volume | 77.8 ± 5.1 | 46.9 ± 12.4 | 109.7 ± 5.7 |
| (μl/beat/100 g | | | |
| body wt) | | | |
| Total Peripheral | 4.22 ± 0.10 | 6.16 ± 0.96 | 2.58 ± 0.10 |
| Resistance | | | |
| (mm Hg/ml/min/100 g | | | |
| body wt) | | | |
| Hemoglobin | 13.7 ± 0.1 | 6.3 ± 0.4 | 5.6 ± 0.1 |
| (g) | | | |
| Hematocrit | 42.0 ± 0.2 | 19.8 ± 1.2 | 17.6 ± 0.2 |
| (%) | | | |
| Maximum | N.A. | 13.0 ± 0.7 | 12.2 ± 0.7 |
| Hemorrhage Volume | | | |
| (mls) | | | |

TABLE 2
Alterations in Regional Blood Flow at 2 h after T-H and Resuscitation

| Assessment Parameter | | | |
|---|---|---|---|
| (ml/min/100 g tissue) | SHAM | T-H | G-T-H |
| Portal Vein | 205 ± 26 | 97 ± 46 | 187 ± 14 |
| Hepatic Artery | 12.2 ± 5.1 | 13.4 ± 1.8 | 18.5 ± 6.0 |
| Total Hepatic | 217 ± 21 | 110 ± 44 | 202 ± 20 |
| Small Intestine | 202 ± 14 | 115 ± 44 | 240 ± 24 |
| Renal | 627 ± 18 | 178 ± 78 | 374 ± 13 |
| Cardiac | 420 ± 60 | 410 ± 80 | 1,320 ± 170 |
| Splenic | 122 ± 1 | 61 ± 29.51 | 46 ± 20 |
| Thymus | 56.0 ± 16.9 | 29.8 ± 9.2 | 47.8 ± 10.5 |
| Brain | 58 ± 2 | 57 ± 22 | 103 ± 11 |
| Lung | 48.1 ± 1.8 | 21.8 ± 15.4* | 74.2 ± 18.1 |
| Large Intestine | 46 ± 4 | 46 ± 17 | 101 ± 18 |
| Gastric | 76.7 ± 2.0 | 27.5 ± 8.9 | 78.3 ± 5.1 |
| Pancreatic | 120 ± 6 | 56 ± 35 | 56 ± 4 |
| Skin | 43.3 ± 12.2 | 15.1 ± 4.4 | 53.9 ± 5.7 |
| Muscle | 5.24 ± 0.74 | 2.21 ± 1.06 | 6.45 ± 1.69 |
| Mesenteric | 53.7 ± 31.1 | 23.7 ± 10.0 | 29.2 ± 5.0 |
| Cecum | 149 ± 25 | 61 ± 28 | 118 ± 29 |

*One animal excluded from analyses due to aberrantly high value (287.1)

The effects of glucosamine were astounding. Glucosamine improved outcome in the rat model of trauma-hemorrhage and resuscitation. Cardiac output improved more than two-fold over the T-H group and more than 50% over the sham group. Relative to the T-H group, in the G-T-H group oxygen delivery and oxygen consumption more than doubled, and both +dp/dt and −dp/dt markedly improved. Total peripheral resistance was decreased. Hepatic blood flow improved, as did distribution to most of the organs assessed.

### Example 2

A second experiment was performed in order to mimic a trauma/hemorrhage in the field. There were no treatments in any of the three groups prior to trauma/hemorrhage. The T-H and G-T-H groups were partially resuscitated identically with two times the maximum hemorrhage volume over a period of 30 minutes. In the G-T-H group, a 1 ml bolus containing an iso-osmotic solution of 75 mM glucosamine (pH 7.4) in 50% RL was administered, while in the T-H group this bolus contained only RL. This was then followed with two times the maximum hemorrhage volume over a period of 30 minutes with RL (T-H) or RL containing 10 mM glucosamine (G-T-H). Sham-operated animals underwent the same surgical procedure but were neither bled nor resuscitated. The data for this experiment are shown in Tables 3 and 4 (values are mean ±standard error of the mean). As in Example 1, glucosamine was again remarkably protective.

TABLE 3
Alterations in Systemic Hemodynamic Parameters 2 h after T-H

| Assessment Parameter | SHAM (n = 8) | T-H (n = 8) | G-T-H (n = 8) |
|---|---|---|---|
| Cardiac Output | 26.06 ± 1.15 | 12.10 ± 4.28 | 33.49 ± 2.12 |
| (ml/min 100 g body wt) | | | |
| Oxygen Delivery | 4.84 ± 0.27 | 1.08 ± 0.38 | 2.52 ± 0.17 |
| (ml/min 100 g body wt) | | | |
| Oxygen Consumption | 1.96 ± 0.16 | 0.61 ± 0.22 | 0.93 ± 0.23 |
| (ml/min 100 g body wt) | | | |
| Oxygen Extraction | 40.83 ± 3.44 | 58.95 ± 20.84 | 34.48 ± 7.45 |
| (%) | | | |
| Heart Rate | 415.50 ± 6.37 | 316.50 ± 111.90 | 394.50 ± 13.13 |
| (beat/minute) | | | |
| Mean Arterial Pressure | 117.75 ± 3.46 | 79.50 ± 28.11 | 97.88 ± 4.19 |
| (mm Hg) | | | |
| +dp/dt | 16170 ± 1060 | 10628 ± 3757 | 13656 ± 7786 |
| (mm Hg/sec) | | | |
| −dp/dt | 10272 ± 933 | 5066 ± 1791 | 8549 ± 549 |
| (mm Hg/sec) | | | |
| Stroke Volume | 62.83 ± 2.99 | 37.20 ± 13.15 | 84.95 ± 4.66 |
| (μl/beat/100 g body wt) | | | |
| Total Peripheral Resistance | 4.58 ± 0.23 | 7.93 ± 2.80 | 3.05 ± 0.31 |
| (mm Hg/ml/min/100 g body wt) | | | |
| Hemoglobin | 14.00 ± 0.20 | 6.49 ± 2.29 | 5.31 ± 0.26 |
| (g) | | | |
| Hematocrit | 42.90 ± 0.61 | 20.41 ± 7.22 | 16.74 ± 0.76 |
| (%) | | | |
| Maximum Hemorrhage Volume | 332.50 ± 12.94 | 294.38 ± 104.08 | 277.38 ± 10.24 |
| (mls) | | | |

TABLE 4
Alterations in Regional Blood Flow at 2 h after T-H and Resuscitation

| Assessment | | | |
|---|---|---|---|
| Parameter (ml/min/ | | | |
| 100 g tissue) | SHAM (n = 8) | T-H (n = 8) | G-T-H (n = 8) |
| Portal Vein | 116.23 ± 15.80 | 79.28 ± 15.42 | 211.57 ± 23.79 |
| Hepatic Artery | 16.76 ± 2.93 | 25.98 ± 6.10 | 45.10 ± 6.58 |
| Total Hepatic | 132.99 ± 14.74 | 105.26 ± 20.60 | 256.67 ± 27.12 |
| Small Intestine | 144.06 ± 22.76 | 86.07 ± 17.19 | 233.60 ± 27.07 |
| Renal | 493.71 ± 28.82 | 169.91 ± 40.26 | 472.07 ± 49.40 |
| Cardiac | 553.50 ± 78.68 | 436.04 ± 72.36 | 933.65 ± 114.73 |
| Splenic | 53.93 ± 11.64 | 18.87 ± 5.67 | 54.77 ± 4.44 |
| Thymus | 56.46 ± 7.97 | 27.78 ± 8.69 | 40.70 ± 4.96 |
| Brain | 60.85 ± 4.79 | 55.83 ± 7.41 | 151.58 ± 13.97 |
| Lung | 43.46 ± 10.69 | 32.14 ± 11.50 | 29.71 ± 4.87 |
| Large Intestine | 45.73 ± 4.23 | 28.96 ± 4.89 | 96.83 ± 6.90 |
| Gastric | 69.34 ± 8.44 | 31.21 ± 7.65 | 71.08 ± 6.84 |
| Pancreatic | 116.96 ± 24.35 | 21.71 ± 4.56 | 52.08 ± 5.53 |
| Skin | 10.24 ± 1.68 | 3.23 ± 0.61 | 11.65 ± 1.25 |
| Muscle | 16.50 ± 2.51 | 6.09 ± 1.02 | 32.97 ± 5.00 |
| Mesenteric | 35.98 ± 3.51 | 13.96 ± 3.77 | 43.12 ± 9.56 |
| Cecum | 100.41 ± 14.31 | 40.03 ± 12.03 | 86.39 ± 10.70 |

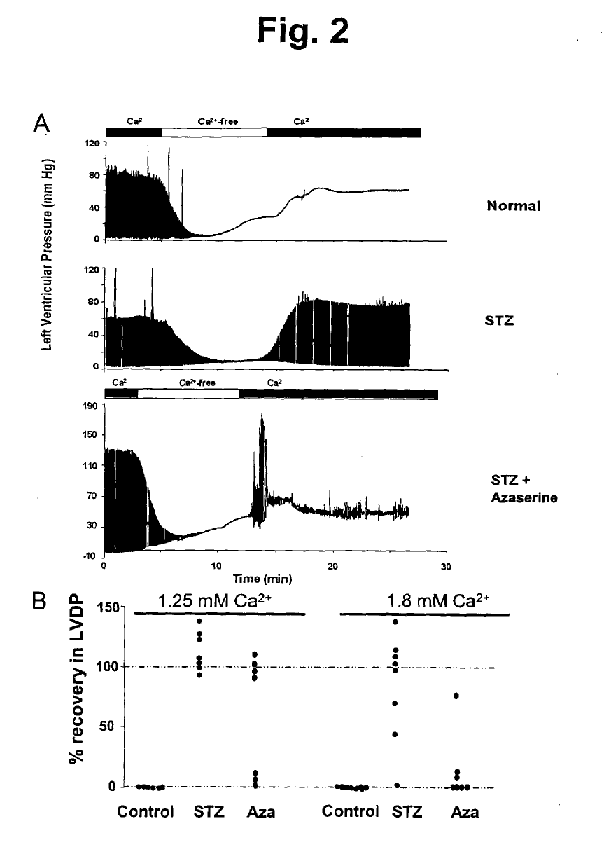

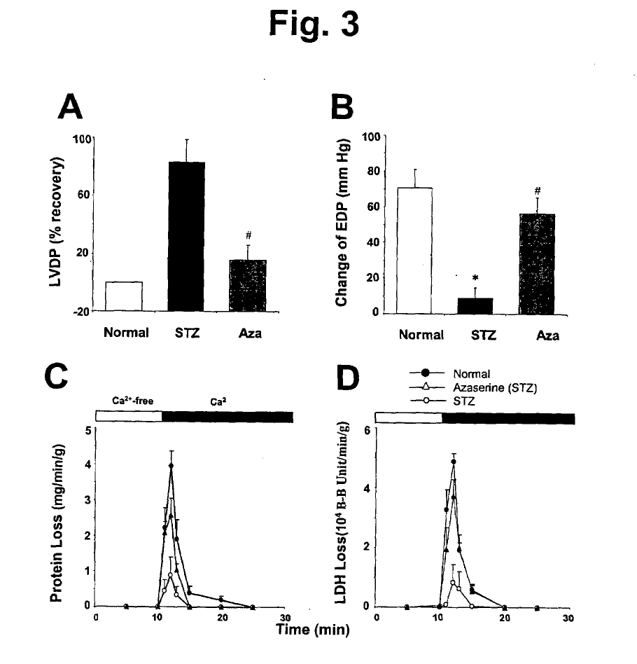

The effect of short-term hyperglycemia (2-5 days) on the calcium paradox was explored. The protocol (a 30-minute stabilization after hanging the heart on a Langendorff apparatus, a Ca²⁺-free buffer for 10 min, a buffer containing 1.25 mM Ca²⁺ buffer for 15 min) was initially applied to perfused hearts from control rats. When calcium was removed from the perfusate, the heart gradually ceased beating (FIG. 2A), although no protein loss from the heart was detected (FIG. 3C). Upon readdition of calcium, an increase in left ventricular pressure (LVP) was apparent, although beating did not resume. The decrease in left ventricular diastolic pressure (LVDP) and an elevation in end diastolic pressure (EDP), as well as protein loss and blanching signaled the transition to a “stone heart” (Zimmerman, Cardiovasc Res (2000) 45(1):119-121) in hearts from all control animals examined. In contrast, hearts from 7 of 7 animals made hyperglycemic by treatment with streptozotocin (STZ), even for only several days duration, demonstrated almost complete protection in the calcium paradox.

Azaserine (AZA), the inhibitor of GFAT and HBP flux (Marshall, et al., J Biol Chem (1991) 266(8):4706-4712), negated the effects of hyperglycemia, restoring sensitivity to the paradox in 3 of 7 hearts (FIG. 2B). Because in these experiments, hearts from the STZ animals actually performed better after calcium readdition than before, the perfusate Ca²⁺ was increased from 1.25 mM to 1.8 mM to accentuate the Ca²⁺ overload. This decreased somewhat the protection seen with hyperglycemia, but made the reversal seen with azaserine more pronounced (FIGS. 2B and 3). These data show that glucosamine and other CCE inhibitors preserve function in the calcium paradox.

### Example 4

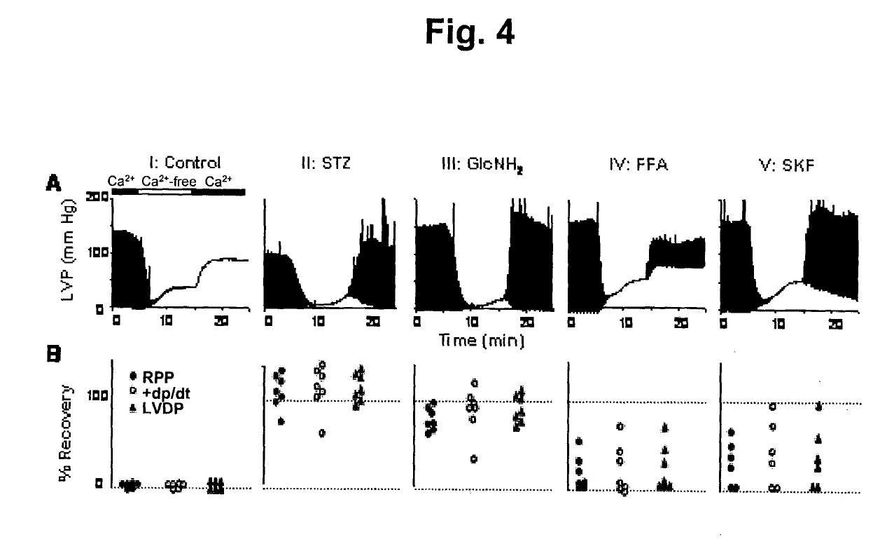

To confirm the involvement of the HBP in the protection due to hyperglycemia, a complementary series of experiments at a final Ca²⁺ concentration of 1.25 mM were performed. A 5-minute pretreatment of control hearts with glucosamine did not alter function prior to calcium removal, and afforded significant protection in 7 of 7 hearts following calcium readdition (FIG. 4). Protein loss was also inhibited by 88% (data not shown).

An independent mechanism of increasing metabolites in the HBP is to provide the heart with free fatty acids (Hawkins, et al., J Clin Invest (1997) 99(9):2173-2182). This occurs because of the “glucose-sparing” effect, a rapid block on the entry of glucose into the glycolytic pathway because of an inhibition of phosphofructokinase. A 45-minute pretreatment with hexanoate, a short-chain free fatty acid, resulted in substantive protection in 3 of 7 animals. In addition, SKF96365 (also known as 1-[β-[3-(4-methoxyphenyl)propoxy]-4-methoxyphenethyl]-1H-imidazole, HCl; available from Calbiochem, San Diego, Calif.) as an independent means of blocking the channels responsible for CCE was utilized (Putney, Capacitative Calcium Entry (1997) Austin: Landes Biomedical). This too was at least partially effective in 4 of 6 animals (FIG. 4).

### Example 5

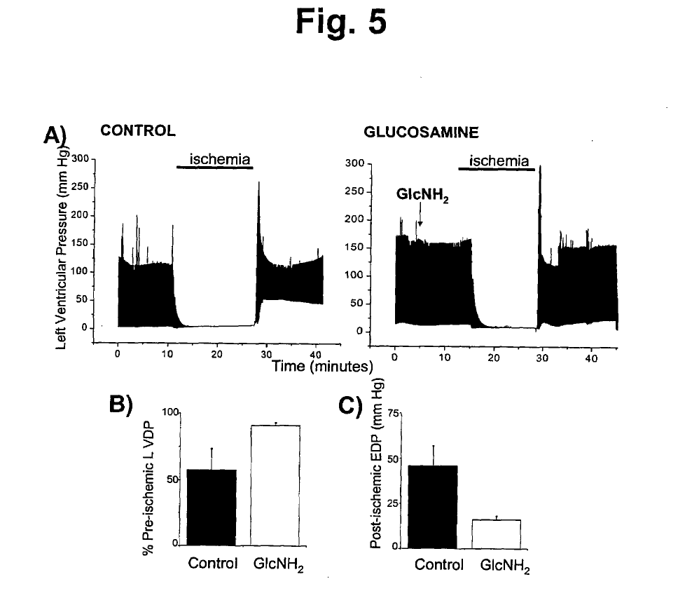

To determine whether glucosamine treatment was protective in an isolated heart model of ischemia and reperfusion, isolated rat hearts (n=3) were assessed for protection against damage utilizing an ischemia/reperfusion protocol that included 10 minutes of global ischemia followed by 15 minutes of reperfusion with oxygenated buffer. In the experimental group, 5 mM glucosamine was added to the buffer 10 minutes prior to the onset of ischemia and was also included in the reperfusion buffer. As shown in FIG. 5A, this brief glucosamine treatment was able to provide significant protection due to ischemia/reperfusion, as assessed by both LVDP (FIG. 5B) and EDP FIG. 5C).

### Example 6

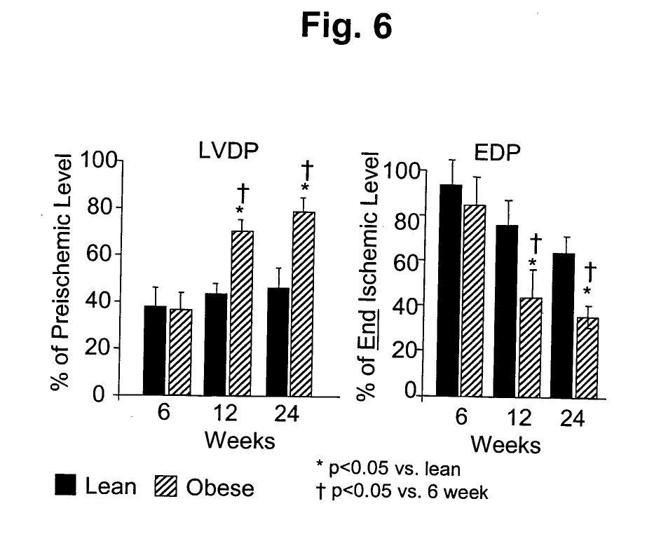

Hearts isolated from a model of Type II diabetes, Zucker fa/fa rats were examined for their response to an ischemia/reperfusion protocol that included 30 minutes of low flow during the ischemic phase. The experiment was carried out with both obese Zucker fa/fa and lean (either FA/FA or FA/fa) rats of six, twelve, and twenty four weeks of age. At six weeks, the fa/fa animals are hyperinsulinemic but not yet hyperglycemic. As seen by other investigators (Feuvray and Lopaschuk, Cardiovasc Med (1997) 2:152-155; Nawata, et al., J Cardiovasc Pharmacol (2002) 40(4):491-500; Ravingerova, et al., Mol Cell Biochem (2000) 210(1-2):143-151; and Hadour, et al., J Mol Cell Cardiol (1998) 30(9): 1869-1875), the hyperglycemic groups were protected significantly from damage due to ischemia/reperfusion, as assessed by both LVDP and EDP (FIG. 6). Interestingly, the six-week old fa/fa rats were not protected, although they were hyperinsulinemic, implicating a role for hyperglycemia in the protection.

### Example 7

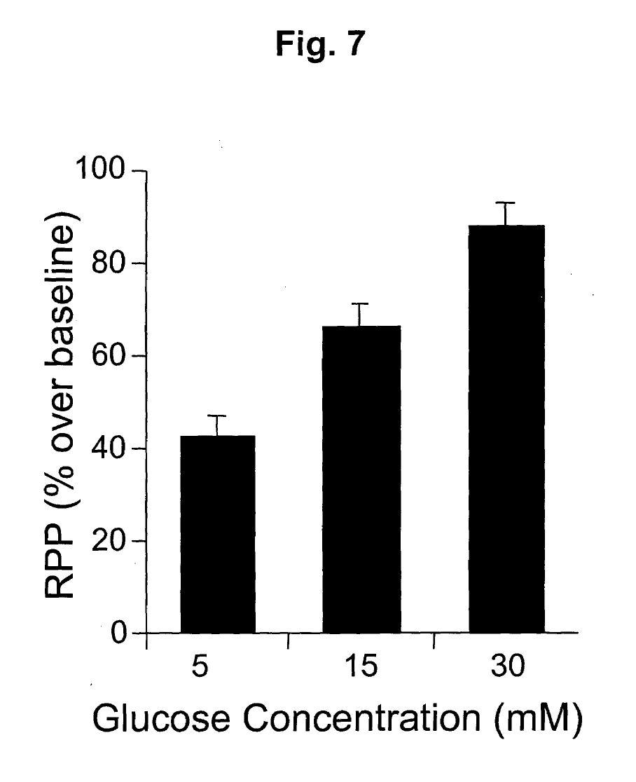

The effects of varying glucose concentrations in the presence of high insulin (1000 μU/ml) were also assessed. Using a low-flow isolated rat heart model of ischemia, heart function was assessed 30 minutes following low flow perfusion of insulin and 5, 15, or 30 mM glucose. As seen in FIG. 7, the greatest degree of recovery of heart function was observed in hearts exposed to both high insulin and high glucose.

### Example 8

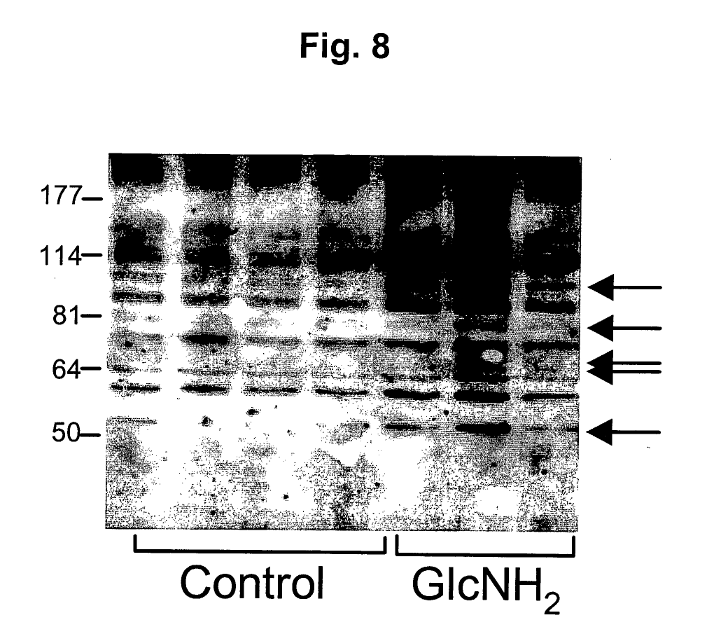

In the experiments above with isolated hearts, glucosamine was protective after only a ten minute pre-incubation. While not wishing to be bound by theory, the mechanism believed to be responsible for the protection is an increase in concentration of an intracellular metabolite of the HBP, which results in an increase in protein-associated O-GlcNAc. Therefore, the following experiments demonstrated whether (1) such short incubations with glucosamine are able to increase pools of UDP-GlcNAc and (2) allow for additional O-GlcNAc modifications of cellular proteins. With respect to the first point, it has been demonstrated that 30 minute incubations with 5 mM glucosamine in isolated hearts give rise to about 75% increases in UDP-GlcNAc pools. Increases at ten minutes were 47%. With respect to the second point, experiments were performed in which a monoclonal antibody specific for O-GlcNAc on proteins (CTD110) (Corner, et al, Anal Biochem (2001) 293(2):169-177) was used in an immunoblotting protocol. The specificity of this antibody for O-GlcNAc-containing epitopes is verified in each experiment by the inclusion of 10 mM GlcNAc along with the CTD110 antibody on parallel samples. These samples show no staining (data not shown). CTD110 immunoblots were performed on extracts from isolated hearts following a ten-minute perfusion with buffer or buffer containing 5 mM glucosamine (FIG. 8). Clear differences in the O-GlcNAc-containing protein pattern are evident (arrows), although there was some sample-to-sample variation. Intriguingly, one group of newly appearing proteins has molecular masses near 70 kDa and another around 90 kDa. Walgren et al. (Walgren, et al., Am J Physiol Endocrinol Metab (2003) 284(2):E424-E434) found that one of the proteins that displayed increased O-GlcNAc following high glucose and insulin in L6 myocytes was HSP-70. Another family of heat shock proteins, HSP-90, has a molecular mass near 90 kDa (Nollen and Morimoto, J Cell Sci (2002) 115(Pt 14):2809-2816). Thus, O-GlcNAc bearing proteins appear to be responsible for the protection.

Analysis

These examples illustrate that the methods disclosed herein can effectively intervene so as to minimize injury and promote cell viability and/or healing during stress. The examples demonstrate that in rat and pig models of hypovolemic stress, the infusion of compositions such as glucosamine can lead to a striking improvement in post-trauma function. Also, the examples demonstrate that glucosamine is highly protective in isolated heart models of ischemia/reperfusion and calcium overload. While not wishing to be bound by theory, it is proposed that this protection results from an amplification of a naturally occurring, stress-activated, pro-survival pathway that is characterized by increasing a concentration of an intracellular metabolite of the HBP, which can lead to increased levels of the nucleotide sugar UDP-GlcNAc, the substrate for a glycosylation reaction that is now well-characterized but highly atypical (Wells, et al., Science (2001) 291(5512):2376-2378). Elevation in the levels of protein-associated O-GlcNAc is believed to be a critical adaptive response that increases the chances of survival of both the organism and the cell during and following periods of stress. In addition, the methods described herein that amplify and/or accelerate increases in intracellular metabolites of the HBP in cells, tissue, or organ, greatly decrease the cellular and tissue damage that would otherwise result from a stress, in particular hypovolemic stress and ischemia/reperfusion injury. As a corollary, one means of achieving this protection occurs because an increase in a concentration of an intracellular metabolite of the HBP can inhibit the calcium overload that can result from stress and often leads to cell death.

The data demonstrate a remarkable protective effect of compositions such as glucosamine in preserving cardiac function in models of hypovolemic stress in rats as well as in different models of cardiac injury in the isolated heart, such as the calcium paradox and ischemia/reperfusion injury. Furthermore, the data demonstrate that the protection is likely mediated at least in part via increased O-GlcNAc and subsequent inhibition of calcium influx.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of reducing a pathogenic effect caused by stress in a subject, comprising administering to the subject a composition that increases a concentration of an intracellular metabolite of a hexosamine biosynthetic pathway, as compared to the concentration of the intracellular metabolite in the absence of the composition, the increase in the concentration of the intracellular metabolite of the hexosamine biosynthetic pathway reducing the pathogenic effect of the stress in the subject.

2. The method of claim 1, wherein the stress is not associated with a hyperactivated inflammatory response.

3. The method of claim 1, wherein the increase in the concentration of the intracellular metabolite inhibits cellular calcium overload.

4. The method of claim 1, wherein the intracellular metabolite is uridine diphosphate-N-acetylglucosamine.

5. The method of claim 1, wherein the composition comprises an inhibitor of O—N-acetyl glucosaminease (O-GlcNAcase).

6. The method of claim 5, wherein the inhibitor comprises O-(2-acetamido-2-deoxy-D-glucopyranosylidene)amino-N-phenylcarbamate (PUGNAc).

7. The method of claim 1, wherein the composition comprises glucosamine, N-acetylglucosamine, or a pharmaceutically acceptable salt or a polymer thereof.

8. The method of claim 1, wherein the composition comprises glutamine or a pharmaceutically acceptable salt thereof.

9. The method of claim 1, wherein the composition comprises fructose-1,6-bisphosphate or a pharmaceutically acceptable salt thereof.

10. The method of claim 1, wherein the composition comprises any combination of glucosamine, N-acetylglucosamine, glutamine, fructose-1,6-bisphosphate, O-(2-acetamido-2-deoxy-D-glucopyranosylidene)amino-N-phenylcarbamate, or a pharmaceutically acceptable salt or a polymer thereof.

11. The method of claim 1, wherein the composition is administered to the subject prior to, during, or after the stress.

12. The method of claim 1, wherein the stress is caused by ischemia.

13. The method of claim 1, wherein the stress is caused by hemorrhage.

14. The method of claim 1, wherein the stress is caused by hypovolemic shock.

15. The method of claim 1, wherein the stress is caused by myocardial infarction.

16. The method of claim 1, wherein the stress is caused by stroke.

17. The method of claim 1, wherein the stress is caused by a medical procedure.

18. The method of claim 17, wherein the medical procedure is an interventional cardiology procedure.

19. The method of claim 17, wherein the medical procedure is cardiac bypass surgery.

20. The method of claim 17, wherein the medical procedure is fibrinolytic therapy.

21. The method of claim 17, wherein the medical procedure is angioplasty.

22. The method of claim 17, wherein the medical procedure is a stent placement.

23. The method of claim 1, wherein the composition comprises a solution of from about 0.1 mM to about 1 M glucosamine in about 100% to about 50% Ringer's lactate.

24. The method of claim 1, wherein the composition is administered over a period of from about 5 minutes to about 1 hour.

25. The method of claim 1, wherein the subject is a mammal.

26. The method of claim 25, wherein the mammal is a human.

27. A method of preserving a cell, tissue, or organ transplant in a transplant recipient, comprising:
a. contacting the cell, tissue, or organ transplant with a composition that increases a concentration of an intracellular metabolite of a hexosamine biosynthetic pathway, as compared to the concentration of the intracellular metabolite in the absence of the composition; and
b. transplanting the cell, tissue, or organ into the recipient, the increase in the concentration of the intracellular metabolite of the hexosamine biosynthetic pathway preserving the cell, tissue, or organ transplant in the transplant recipient.

28. The method of claim 27, wherein the contacting step is performed prior to the transplantation step.

29. The method of claim 27, wherein the contacting step is performed during the transplantation step.

30. The method of claim 27, wherein the contacting step is performed after the transplantation step.

31. The method of claim 27, wherein the organ transplant is a heart.

32. The method of claim 27, wherein the composition comprises any combination of glucosamine, N-acetylglucosamine, glutamine, fructose-1,6-bisphosphate, O-(2-acetamido-2-deoxy-D-glucopyranosylidene)amino-N-phenylcarbamate, or a pharmaceutically acceptable salt or a polymer thereof.

33. The method of claim 27, wherein the composition comprises glucosamine, glucosamine polymer, or a pharmaceutically acceptable salt thereof.

34. The method of claim 27, wherein the composition comprises glucosamine, glucosamine polymer, or a pharmaceutically acceptable salt thereof in a concentration of from about 0.1 mM to about 1 M.

35. The method of claim 27, wherein the intracellular metabolite is uridine diphosphate-N-acetylglucosamine.

36. The method of claim 35, wherein the concentration of uridine diphosphate-N-acetylglucosamine is increased by about 75%, 30 minutes after contact with the composition.

37. The method of claim 35, wherein the concentration of uridine diphosphate-N-acetylglucosamine is increased by about 50%, 10 minutes after contact with the composition.

38. The method of claim 35, wherein the concentration of uridine diphosphate-N-acetylglucosamine is increased by about 20%, 10 minutes after contact with the composition.

39. The method of claim 27, wherein the intracellular metabolite is glucosamine-6-phosphate.

40. A method of preserving a cell, tissue, or organ culture, comprising contacting the cell, tissue, or organ culture with a composition that increases a concentration of an intracellular metabolite of a hexosamine biosynthetic pathway, as compared to the concentration of the intracellular metabolite in the absence of the composition, the increase in the concentration of the intracellular metabolite of the hexosamine biosynthetic pathway preserving the cell, tissue, or organ culture.

41. The method of claim 40, wherein the composition comprises any combination of glucosamine, N-acetylglucosamine, glutamine, fructose-1,6-bisphosphate, O-(2-acetamido-2-deoxy-D-glucopyranosylidene)amino-N-phenylcarbamate, or a pharmaceutically acceptable salt or a polymer thereof.

42. The method of claim 40, wherein the composition comprises glucosamine, glucosamine polymer, or a pharmaceutically acceptable salt thereof.

43. The method of claim 40, wherein the composition comprises glucosamine, glucosamine polymer, or a pharmaceutically acceptable salt thereof in a concentration of from about 0.1 mM to about 1 M.

44. The method of claim 40, wherein the intracellular metabolite is uridine diphosphate-N-acetylglucosamine.

45. The method of claim 44, wherein the concentration of uridine diphosphate-N-acetylglucosamine is increased by about 75%, 30 minutes after contact with the composition.

46. The method of claim 44, wherein the concentration of uridine diphosphate-N-acetylglucosamine is increased by about 50%, 10 minutes after contact with the composition.

47. The method of claim 44, wherein the concentration of uridine diphosphate-N-acetylglucosamine is increased by about 20%, 10 minutes after contact with the composition.

48. The method of claim 40, wherein the intracellular metabolite is glucosamine-6-phosphate.
